(19) United States
(12) Patent Application Publication
(10) Pub. No.: US 2015/0094210 A1
(43) Pub. Date: Apr. 2, 2015

(54) METHOD, SYSTEM AND COMPUTER READABLE MEDIUM FOR DETERMINING BASE INFORMATION IN PREDETERMINED AREA OF FETUS GENOME

(71) Applicants: Shengpei Chen, Shenzhen (CN); Huijuan Ge, Shenzhen (CN); Xuchao Li, Shenzhen (CN); Shang Yi, Shenzhen (CN); Jian Wang, Shenzhen (CN); Jun Wang, Shenzhen (CN); Huanming Yang, Shenzhen (CN); Xiuqing Zhang, Shenzhen (CN)

(72) Inventors: Shengpei Chen, Shenzhen (CN); Huijuan Ge, Shenzhen (CN); Xuchao Li, Shenzhen (CN); Shang Yi, Shenzhen (CN); Jian Wang, Shenzhen (CN); Jun Wang, Shenzhen (CN); Huanming Yang, Shenzhen (CN); Xiuqing Zhang, Shenzhen (CN)

(73) Assignee: BGI DIAGNOSIS CO., LTD., Shenzhen, Guangdong (CN)

(21) Appl. No.: 14/395,065

(22) PCT Filed: May 14, 2012
(86) PCT No.: PCT/CN2012/075478

§ 371 Date: Oct. 17, 2014

(51) Int. Cl. C12Q 1/68; G06F 19/22; G06F 19/00; G06F 19/18

(52) U.S. Cl. CPC C12Q 1/6883; G06F 19/18; G06F 19/22; G06F 19/345; G06F 19/3431; C12Q 2600/156; C12Q 2600/118
USPC 506/2; 506/36

(57) ABSTRACT

Provided are a method, system and computer readable medium for determining the base information in a predetermined area of a fetus genome, the method comprising following steps: constructing a sequence library for the DNA samples of the fetus genome; sequencing the sequence library to obtain the sequencing result of the fetus, the sequencing result of the fetus comprised of a plurality of sequencing data; and based on the sequencing result of the fetus, determining the base information in the predetermined area according to the hidden Markov model in conjunction with the genetic information of an individual related hereditarily to the fetus.

### TECHNICAL FIELD

Embodiments of the present disclosure generally relate to a method of determining base information of a predetermined region in a fetal genome, and a system and a computer readable medium thereof.

### BACKGROUND

Genetic diseases are one kind of diseases caused by changes of genetic materials, having characteristics of being congenital, familial, permanent and hereditary. The genetic diseases may be categorized into 3 classes: monogenetic disease, polygenetic disorder and chromosome abnormality. In which the monogenetic disease is mostly because of genetic function abnormality caused by dominant or recessive inheritance of a single disease-causing gene; while the polygenetic disorder is a kind of disease caused by a plurality of gene changes, which may be influenced by external environment to some extent; and the chromosome abnormality includes number abnormality and structure abnormality, with a most common example being as a Down's Syndrome resulting from Trisomy 21, of which a child patient presenting congenital traits such as mongolism and abnormal body shape, etc. Since there are no effective therapeutic treatments for genetic diseases so far, it can only pertinently perform supportive treatments or drug remission with expensive cost, which may bring heavy burdens both in economy and spirit for societies and families. Thus, it is extremely necessary to do some preventive work by detecting pathological status with a fetus before birth, to achieve a purpose of good prenatal and postnatal care.

However, related detection method still needs to be improved.

### SUMMARY

Embodiments of the present disclosure seek to solve at least one of the problems existing in the related art to at least some extent.

Embodiments of a first broad aspect of the present disclosure provide a method of determining base information of a predetermined region in a fetal genome. According to embodiments of the disclosure, the method may comprise: constructing a sequencing library based on a genomic DNA sample of a fetus; subjecting the sequencing library to sequencing, to obtain a sequencing result of the fetus consisting of a plurality of sequencing data; and determining the base information of the predetermined region based on the sequencing result of the fetus combining with genetic information of a related individual using a hidden Markov Model. A formation of offspring genome equals to a random recombination with parental generation's genome (i.e., an interchange of haplotype recombination, and a random combination of gametes). For pregnant plasma, if a fetal haplotype (a recombination of parental haplotypes) is assumed as hidden states, sequencing data of the plasma may be used as observations (observing sequence), transition probabilities, observation symbol probabilities and initial state distribution may be deduced in virtue of prior data, then the most possible fetal haplotype recombination may be determined using a hidden Markov Model based on Viterbi algorithm, so as to obtain more information of fetus prior to birth. Thus, according to embodiments of the present disclosure, in virtue of the hidden Markov Model, for example using the Viterbi algorithm, and referring to genetic information of a related individual, nucleic acid sequence of a predetermined region in a fetal genome may be determined, by which a prenatal genetic detection may be effectively performed with genetic information of fetal genome.

Embodiments of a second broad aspect of the present disclosure provide a system for determining base information of a predetermined region in a fetal genome. According to embodiments of the present disclosure, the system may comprise: a library constructing apparatus, adapted for constructing sequencing library based on a genomic DNA sample of a fetus; a sequencing apparatus, connected to the library constructing apparatus, and adapted for subjecting the sequencing library to sequencing, to obtain a sequencing result of the fetus consisting of a plurality of sequencing data; and an analyzing apparatus, connected to the sequencing apparatus, and adapted for determining the base information of the predetermined region based on the sequencing result of the fetus combining with genetic information of a related individual using a hidden Markov Model. Using the system may effectively implement the above method of determining base information of a predetermined region in a fetal genome, which may determine nucleic acid sequence of a predetermined region in a fetal genome may be determined in virtue of the hidden Markov Model, for example using the Viterbi algorithm, and referring to genetic information of a related individual, by which a prenatal genetic detection may be effectively performed with genetic information of the fetal genome.

Embodiments of a third broad aspect of the present disclosure provide a computer readable medium. According to embodiments of the present disclosure, the computer readable medium including a plurality of instructions is adapted for determining base information of a predetermined region based on a sequencing result of a fetus combining with genetic information of a related individual using a hidden Markov Model. Using the computer readable medium of the present disclosure may effectively execute the plurality of instructions by a processor, to determine a nucleic acid sequence of the predetermined region in the fetal genome in virtue of the hidden Markov Model, for example using the Viterbi algorithm based on the sequencing data of the fetus combining with genetic information of a related individual, by which prenatal genetic detection may be effectively performed with genetic information of the fetal genome.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference the accompanying drawings, in which:

FIG. 1 is a flow chart showing an analyzing process using a hidden Markov Model according to an embodiment of the present disclosure; and

FIG. 2 is a schematic diagram showing a system for determining base information of a predetermined region in a fetal genome according to an embodiment of the present disclosure.

### DETAILED DESCRIPTION

Reference will be made in detail to embodiments of the present disclosure. The same or similar elements and the elements having same or similar functions are denoted by like reference numerals throughout the descriptions. The embodiments described herein with reference to drawings are explanatory, illustrative, and used to generally understand the present disclosure. The embodiments shall not be construed to limit the present disclosure.

It should note that terms such as “first” and “second” are used herein for purposes of description and are not intended to indicate or imply relative importance or significance. Thus, features defined with “first”, “second” may explicitly or implicitly include one or more the features. Furthermore, in the description of the present disclosure, unless otherwise stated, “a/the plurality of” means two or more.

Method of Determining Base Information of a Predetermined Region in a Fetal Genome

In a first aspect of the present disclosure, there is provided a method of determining base information of a predetermined region in a fetal genome. According to embodiments of the present disclosure, the method may comprise:

firstly, constructing a sequencing library based on a genomic DNA sample of a fetus. According to embodiments of the present disclosure, source of the genomic DNA sample of the fetus is not subjected to any special restrictions. According to some embodiments of the present disclosure, any pregnant samples containing a nucleic acid of a fetal may be used. For example, according to embodiments of the present disclosure, the pregnant sample may be breast milk, urine and peripheral blood from a pregnant woman. In which, the pregnant peripheral blood is preferred. Using the pregnant peripheral blood as the source of the genomic DNA sample of the fetus may effectively realize obtaining the genomic DNA sample of the fetus by noninvasive sampling, by which the fetal genome may be effectively monitored in the premise of having no influence on normal development of fetal growth. As for methods and processes of constructing a sequencing library for the nucleic acid sample, a person skilled in the art may appropriately select depending on different sequencing technology. Detailed process may refer to procedure provided by sequencer manufacturer, such as Illumina Company, for example, refer to Multiplexing Sample Preparation Guide (Part #1005361; February 2010) or Paired-End SamplePrep Guide (Part #1005063; February 2010) from Illumina Company, which are incorporated herein for reference. According to embodiments of the present disclosure, methods and devices for extracting a nucleic acid from a biological sample are not subjected to any special restrictions, which may be performed using a commercial nucleic acid extracting kit.

After being constructed, obtained sequencing library is applied to a sequencer, to obtain a corresponding sequencing result consisting of a plurality of sequencing data. According to embodiments of the present disclosure, methods and devices for sequencing are not subjected to any special restrictions, including but not limited to Chain Termination Method (Sanger); a high-throughput sequencing method is preferred. Thus, using characteristics being high-throughput and deep sequencing of these apparatus, efficiency may be further improved, by which precise and accuracy of subsequent analysis with sequencing data, such as statistical test, may be further improved. The high-throughput sequencing method includes but not limited to a Next-Generation sequencing technology or a single sequencing technology. The Next-Generation sequencing platform (Metzker M L. Sequencing technologies-the next generation. Nat Rev Genet. 2010 January; 11(1):31-46) includes but not limited to Illumina-Solexa (GATM, HiSeq2000™, etc), ABI-Solid and Roche-454 (pyrosequencing) sequencing platform; the single sequencing platform (technology) includes but not limited to True Single Molecule DNA sequencing of Helicos Company, single molecule real-time (SMRT™) of Pacific Biosciences Company, and nonapore sequencing technology of Oxford Nanopore Technologies (Rusk, Nicole (Apr. 1, 2009). Cheap Third-Generation Sequencing. Nature Methods 6 (4): 244-245), etc. With gradual development of sequencing technology, a person skilled in the art may understand other sequencing methods and apparatuses may also be used for whole genome sequencing. According to specific examples of the present disclosure, the whole genome sequencing library may be subjected to sequencing by at least one selected from Illumina-Solexa, ABI-SOLiD, Roche-454 and a single molecule sequencing apparatus.

Optionally, after being obtained, the sequencing result may be aligned to a reference sequence, to determine sequencing data corresponding to the predetermined region. Term of “predetermined region” used herein should be broadly understood, referring to any region of a nucleic acid molecule containing a possible predetermined event. For SNP analysis, it may be a region containing SNP site. For analyzing chromosome aneuploidy, the predetermined region refers to entire or part of the chromosome to be analyzed, i.e., selecting sequencing data deriving from the chromosome. Methods of selecting sequencing data deriving from a corresponding region in the sequencing result are not subjected to any special restrictions. According to embodiments of the present disclosure, all obtained sequencing data may be aligned to a reference sequence with a known nucleic acid, to obtain the sequencing data deriving from the predetermined region. In addition, according to embodiments of the present disclosure, the predetermined region may also be a plurality of dispersal points which are not discontinuous in a genome. According to embodiments of the present disclosure, a type of used reference sequence may be not subjected to any special restrictions, which may be any known sequences contained a target region. According to embodiments of the present disclosure, the reference sequence may use a known human reference genome. For example, according to embodiments of the present disclosure, the human reference genome is NCBI 36.3, HG18. In addition, according to embodiments of the present disclosure, alignment methods are not subjected to any special restrictions. According to specific examples, SOAP may be used for alignment.

Then, determining a part of a nucleic acid sequence of the predetermined region based on sequencing data corresponding to the predetermined region; and determining other parts of the nucleic acid sequence based on determined part of the nucleic acid sequence of the predetermined region using Viterbi algorithm, to obtain the nucleic acid sequence of the predetermined region. According to embodiments of the present disclosure, the base information of the predetermined region is determined based on the sequencing result of the fetus combining with genetic information of a related individual using a hidden Markov Model. According to embodiments of the present disclosure, the base information of the predetermined region is determined using the hidden Markov Model is performed based on Viterbi algorithm. Thus, a prenatal genetic detection may be effectively performed with genetic information of fetal genome.

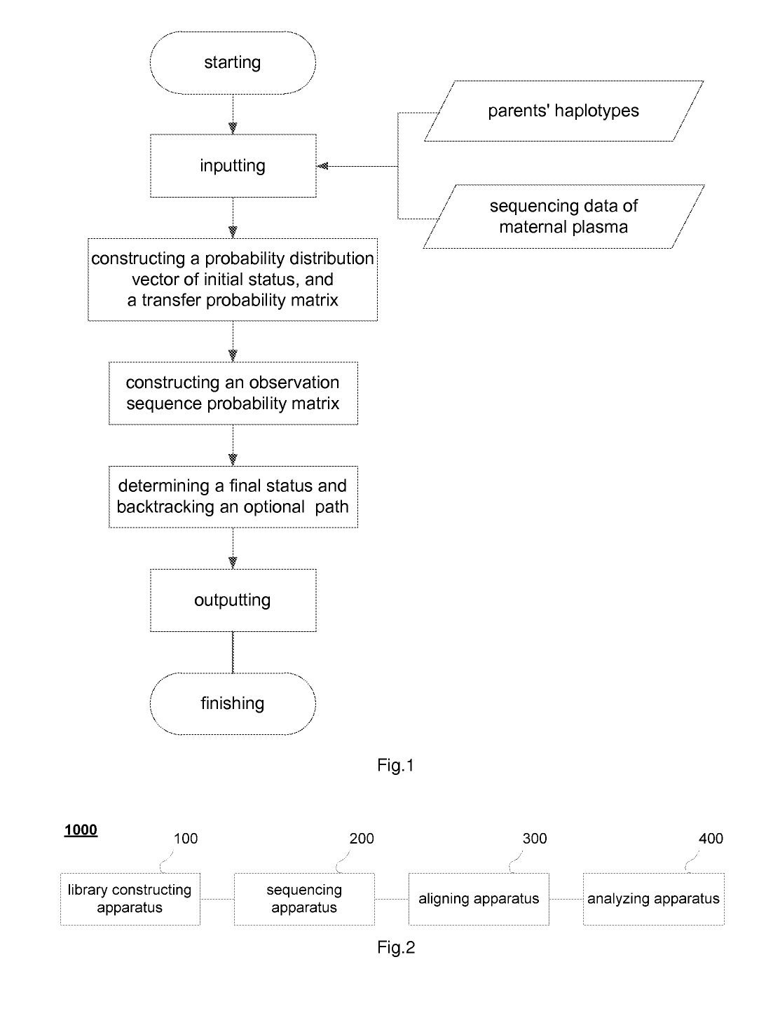

Referring to FIG. 1, a principal for analysis using Viterbi algorithm in virtue of a hidden Markov Model is descripted in details below:

In the genetic sense, term of “a related individual” refers to individuals having a genetic relationship with a fetus. For example, according to embodiments of the present disclosure, “a related individual” may be a rental generation of a fetus, such as parents. Thus, a formation of offspring genome equals to a random recombination with parental generation's genome (i.e., an interchange of haplotype recombination, and a random combination of gametes). For pregnant plasma, if a fetal haplotype (a recombination of parental haplotypes) is assumed as hidden states, sequencing data of the plasma may be used as observations (observing sequence), transition probabilities, observation symbol probabilities and initial state distribution may be deduced in virtue of prior data, then the most possible fetal haplotype recombination may be determined using a hidden Markov Model based on Viterbi algorithm, so as to obtain more information of fetus prior to birth.

Steps of analyzing are shown below in details:

### Marker:

I. the number of sites to be detected is N.
II. haplotypes of parents are respectively recorded as FH={fh₀, fh₁} and MH={mh₀,mh₁},
   in which

mhₖ={m_{1,k}, . . . , m_{i,k}, . . . , m_{N,k}}, fhₖ={f_{1,k}, . . . , f_{i,k}, . . . , f_{N,k}},

∀fh_{i,k},mh_{i,k}∈{A,C,G,T},

k∈{0,1}, i=1,2,3, . . . , N.
III. Unknown fetal haplotype is recorded as H={h₀,h₁}, particularly, h₀ and h₁ respectively represent inheriting from mother and father.

h₀={m_{1,x}_{₁}, . . . , m_{i,x}_{ᵢ}, . . . , m_{N,x}_{_{N}}}, h₁={f_{1,y}_{₁}, . . . , f_{i,y}_{ᵢ}, . . . , f_{N,y}_{N}},

in which xᵢ∈{0,1}, yᵢ∈{0,1},
Subscripts xᵢ and yᵢ respectively present sequence pairs, and qᵢ={xᵢ,yᵢ} represents the hidden states which need to be decoded.
While, all hidden states possible presenting constitutes a set Q.
IV. Sequencing data is recorded as S={s₁, . . . , sᵢ, . . . , s_{N}}
in which sᵢ={n_{i,A},n_{i,C},n_{i,G},n_{i,G}} represents sequencing information of a site, containing the number of four bases, A, C, T and G.

V. A mean fetal concentration and a mean sequencing error rate are respectively recorded as ε and e.
Step 1, constructing a probability distribution vector of an initial state and a transition matrix of haplotypes recombination:
I. The probability distribution of the initial states is recorded as π={πⱼ} (j∈Q).

According to embodiments of the present disclosure, under a circumstance of having no reference data, it may assume that

$${π}_{j} = Pr ( {q}_{1} = j ) {=}^{Δ} \frac{1}{4} , ,$$

i.e., possibilities of each hidden state presenting at the first site are equal.
II. According to embodiments of the present disclosure, a probability of haplotype recombination is recorded as pᵣ=re/N, in which re represents a mean times of human gamete recombinations, with a prior data ranging from 25 to 30.
III. According to embodiments of the present disclosure, a transition matrix of haplotypes recombination is recorded as A={aⱼₖ} (j,k∈Q), in which aⱼₖ represents a probability of hidden states transition, i.e.,

$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix} ,\end{matrix}$$

Subscripts xᵢ and yᵢ of fetal haplotypes h₀={m_{1,x}_{₁}, . . . , m_{i,x}_{ᵢ}, . . . , m_{N,x}_{_{N}}} and h₁={f_{1,y}_{₁}, . . . , f_{i,y}_{ᵢ}, . . . , f_{N,y}_{_{N}}} constitute a sequence pair, qᵢ={xᵢ,yᵢ} constitute the hidden states to be encoded. For example, xᵢ=0 represents “in a maternal chromosome, an allele in the corresponding locus is m_{i,0}”.
Step 2, constructing a probability matrix of observations:

According to embodiments of the present disclosure, the probability matrix of observations is recorded as B={b_{i,j}(sᵢ)} (i=1,2,3, . . . , N, j∈Q), in which b_{i,j}(sᵢ) represents “an observed possibility of this sequencing information in a site i, considering maternal haplotype and fetal haplotype (state j, j={xᵢ, yᵢ})”, i.e.,

$$\begin{matrix}{b}_{i , j} ( {s}_{i} ) = Pr ( {s}_{i} \vert {q}_{i} = j , \{ {m}_{0} , {m}_{1} \} ) \\ = \frac{( {n}_{i , A} + {n}_{i , C} + {n}_{i , G} + {n}_{i , T} ) !}{{n}_{i , A} ! {n}_{i , C} ! {n}_{i , G} ! {n}_{i , T} !} \cdot \\ {( {P}_{i , A} )}^{{n}_{i , A}} \cdot {( {P}_{i , C} )}^{{n}_{i , C}} \cdot {( {P}_{i , G} )}^{{n}_{i , G}} \cdot {( {P}_{i , T} )}^{{n}_{i , T}} ,\end{matrix}$$

in which P_{i,base} represents “a possibility of a base in a site i, considering maternal haplotype and fetal haplotype (state j, j={xᵢ, yᵢ})”, i.e.,

$$\begin{matrix}{P}_{i , base} = Pr ( base \vert {q}_{i} = j , \{ {m}_{0} , {m}_{1} \} ) \\ = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \\ \frac{1}{2} ɛ \cdot Δ ( base , {f}_{{y}_{i}} ) ,\end{matrix}$$

in which, an indicator function is

$$Δ ( x , y ) = \{ \begin{matrix}1 - e & x = y \\ e / 3 & x \neq y\end{matrix} .$$

Such step is to perform HMM parameter, calculating a probability distribution of observation in each site b_{i,j}(sᵢ), i.e., calculating a possibility presenting current sequencing data (observations) in the pregnant plasma, assuming different fetal haplotypes in each site.

Step 3, constructing a partial probability matrix, and a reversal cursor (taking an example of constructing a one-dimensional probability matrix):

Definition: partial probability

$${δ}_{i} ( {q}_{i} ) = ( {max}_{{q}_{i - 1} ∈ Q} {δ}_{i} ( {q}_{i} ) \cdot {a}_{{q}_{i - 1} {q}_{i}} ) \cdot {b}_{i , {q}_{i}} ( {s}_{i} ) ,$$

Definition: reversal cursor)

$${Ψ}_{i} ( {q}_{i} ) = {arg max}_{{q}_{i - 1} ∈ Q} {δ}_{i} ( {q}_{i} ) \cdot {a}_{{q}_{i - 1} {q}_{i}} .$$

Terms of “partial probability δᵢ(qᵢ)” and “reversal cursor Ψᵢ(qᵢ)” used herein both follow classic definitions of Viterbi algorithm. Detailed descriptions for the definition of the parameter may refer to Lawrence R. Rabiner, PROCEEDINGS OF THE IEEE, Vol. 77, No. 2, February 1989, which is incorporated herein by reference.

Step 4, determining a final state, and tracing back an optional path

Determination of the final state,

$${q}_{N}^{*} = {arg max}_{{q}_{N} ∈ Q} {δ}_{N} ( {q}_{N} ) .$$

The most possible fetal haplotype qᵢ*=Ψᵢ(qᵢ) (i=1,2,3, . . . , N−1) is obtained by tracing back the optional path based on the reversal curse.

Step 5, Outputting a Result

Thus, the sequence of the fetal genome may be effectively analyzed. Comparing to other existing method of antenatal detection, the method of the present disclosure may have following technical advantages, mainly embodying in accuracy and amount of genetic information obtainable:

1) According to embodiments of the present disclosure, a site to be detected is not limited to a parental site, for a maternal site, i.e., a maternal heterozygous site, whether a fetus inherits a maternal pathopoiesia site may also be detected excellently, with an accuracy up to 95% or more; and a plurality of abnormality types can be detected, which enlarges a range of disease detection.

2) According to embodiments of the present disclosure, information of a plurality of site and diseases may be obtained by one time of sequencing; while those gene sequence, having a low coverage in the pregnant plasma which is not able to be accurately determined only by enhancing sequencing depth, may be obtained by the method of the present disclosure, with an accurate and liable result.

3. According to embodiments of the present disclosure, a plotting with a genetic disease may be performed, some related diseases may be directly deduced with information of other sites, with a large amount of information obtained for one time, which has a more instructive meaning for clinical detection.

In addition, according to embodiments of the present disclosure, the method of determining base information of a predetermined region in a fetal genome, not limited to a certain genetic polymorphic sites such as SNP or STR, is adapted for all genetic polymorphic sites, which may be parallel used for a plurality of sites, to verify each other. Besides applying to antenatal noninvasive detect genomic information of a fetus, achieving a purpose of disease detection, the method of the present disclosure may also be used in noninvasive antenatal paternity identification, i.e., determining an identity of a fetus' father prior birth, providing assistance for disputes involving rearing responsibilities and obligations, property and sexual assault cases, etc.

System for Determining Base Information of a Predetermined Region in a Fetal Genome

In another aspect of the present disclosure, there is provided a system for determining base information of a predetermined region in a fetal genome. According to embodiments of the present disclosure, referring to FIG. 2, the system 1000 may comprises: a library constructing apparatus 100, a sequencing apparatus 200 and an analyzing apparatus 400.

According to embodiments of the present disclosure, the library constructing apparatus 100 is adapted for constructing sequencing library based on a genomic DNA sample of a fetus. According to embodiments of the present disclosure, the sequencing apparatus 200 is connected to the library constructing apparatus 100, and adapted for subjecting the sequencing library to sequencing, to obtain a sequencing result of the fetus consisting of a plurality of sequencing data. According to embodiments of the present disclosure, the system 1000 may also comprise a DNA sample extracting apparatus, adapted for extracting the genomic DNA sample of the fetus from pregnant peripheral blood. Thus, the system may be adapted for noninvasive antenatal detection.

According to embodiments of the present disclosure, optionally, the system may also comprise an aligning apparatus 300. According to embodiments of the present disclosure, the aligning apparatus 300 is connected to the sequencing apparatus 200, and adapted for aligning the sequencing result of the fetus to a reference sequence, to determine sequencing result deriving from the predetermined region. According to embodiments of the present disclosure, methods and devices for sequencing are not subjected to any special restrictions, including but not limited to Chain Termination Method (Sanger); a high-throughput sequencing method is preferred. Thus, using characteristics being high-throughput and deep sequencing of these apparatus, efficiency may be further improved, by which precise and accuracy of subsequent analysis with sequencing data, such as statistical test, may be further improved. The high-throughput sequencing method includes but not limited to a Next-Generation sequencing technology or a single sequencing technology. The Next-Generation sequencing platform (Metzker M L. Sequencing technologies-the next generation. Nat Rev Genet. 2010 January; 11(1):31-46) includes but not limited to Illumina-Solexa (GATM, HiSeq2000™, etc), ABI-Solid and Roche-454 (pyrosequencing) sequencing platform; the single sequencing platform (technology) includes but not limited to True Single Molecule DNA sequencing of Helicos Company, single molecule real-time (SMRT™) of Pacific Biosciences Company, and nonapore sequencing technology of Oxford Nanopore Technologies (Rusk, Nicole (Apr. 1, 2009). Cheap Third-Generation Sequencing. Nature Methods 6 (4): 244-245), etc. With gradual development of sequencing technology, a person skilled in the art may understand other sequencing methods and apparatuses may also be used for whole genome sequencing. According to specific examples of the present disclosure, the whole genome sequencing library may be subjected to sequencing by at least one selected from Illumina-Solexa, ABI-SOLiD, Roche-454 and a single molecule sequencing apparatus. According to embodiments of the present disclosure, a type of used reference sequence may be not subjected to any special restrictions, which may be any known sequences contained a target region. According to embodiments of the present disclosure, the reference sequence may use a known human reference genome. For example, according to embodiments of the present disclosure, the human reference genome is NCBI 36.3, HG18. In addition, according to embodiments of the present disclosure, alignment methods are not subjected to any special restrictions. According to specific examples, SOAP may be used for alignment.

According to embodiments of the present disclosure, the analyzing apparatus 400 is connected to the sequencing apparatus, and adapted for determining the base information of the predetermined region based on the sequencing result of the fetus combining with genetic information of a related individual using a hidden Markov Model.

According to embodiments of the present disclosure, in the Viterbi algorithm, 0.25 is used as a probability distribution of an initial status, re/N is used as a recombination probability, with re being 25˜30, preferably re being 25, and N being a length of the predetermined region,

$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix}\end{matrix}$$

is used as a recombination transition matrix with pᵣ being re/N.

According to embodiments of the present disclosure, the aligning apparatus is adapted for determining a base having the highest probability based on a formula of

$${P}_{i , base} = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \frac{1}{2} ɛ \cdot Δ ( base , {f}_{{y}_{i}} )$$

$$wherein$$

$$Δ ( x , y ) = \{ \begin{matrix}1 - e & x = y \\ e / 3 & x \neq y\end{matrix} .$$

Analysis with sequencing data, which is detailed descripted above, is also adapted to the system for determining base information of a predetermined region in a fetal genome, which is omitted for brevity.

Thus, using the system may effectively implement the above method of determining base information of a predetermined region in a fetal genome, which may determine nucleic acid sequence of a predetermined region in a fetal genome may be determined in virtue of the hidden Markov Model, for example using the Viterbi algorithm, and referring to genetic information of a related individual, by which a prenatal genetic detection may be effectively performed with genetic information of the fetal genome.

In addition, according to embodiments of the present disclosure, the predetermined region is a site previously determined as having a genetic polymorphism, and the genetic polymorphism is at least one selected from single nucleotide polymorphism and STR.

Terms of “connected” should be broadly understood, which may refer to a direct connection or indirect connection, as long as achieving the above functional connection.

It should note that a person skilled in the art may understand that features and advantages of the method of determining base information of a predetermined region in a fetal genome described above may also adapted to the system for determining base information of a predetermined region in a fetal genome, which are omitted for brevity.

Computer Readable Medium

In a further aspect of the present disclosure, there is provided a computer readable medium. According to embodiments of the present disclosure, the computer readable medium includes a plurality of instructions, adapted for determining base information of a predetermined region based on a sequencing result of a fetus combining with genetic information of a related individual using a hidden Markov Model. Thus, using the computer readable medium may effectively implement the above method of determining base information of a predetermined region in a fetal genome, which may determine nucleic acid sequence of a predetermined region in a fetal genome may be determined in virtue of the hidden Markov Model, for example using the Viterbi algorithm, and referring to genetic information of a related individual, by which a prenatal genetic detection may be effectively performed with genetic information of the fetal genome.

According to embodiments of the present disclosure, the plurality of instructions are adapted for determining the base information of the predetermined region using the hidden Markov model based on Viterbi algorithm. According to embodiments of the present disclosure, in the Viterbi algorithm, 0.25 is used as a probability distribution of an initial status, re/N is used as a recombination probability, with re being 25˜30, preferably re being 25, and N being a length of the predetermined region,

$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix}\end{matrix}$$

is used as a recombination transition matrix with pᵣ being re/N.

According to embodiments of the present disclosure, the plurality of instructions are further adapted for determining a base having the highest probability based on based on a formula of

$${P}_{i , base} = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \frac{1}{2} ɛ \cdot Δ ( base , {f}_{{y}_{i}} )$$

$$wherein$$

$$Δ ( x , y ) = \{ \begin{matrix}1 - e & x = y \\ e / 3 & x \neq y\end{matrix} .$$

Analysis with sequencing data, which is detailed descripted above, is also adapted to the computer readable medium, which is omitted for brevity.

In addition, according to embodiments of the present disclosure, the predetermined region is a site previously determined as having a genetic polymorphism, and the genetic polymorphism is at least one selected from single nucleotide polymorphism and STR.

As to the specification, “computer readable medium” may be any device adaptive for including, storing, communicating, propagating or transferring programs to be used by or in combination with the instruction execution system, device or equipment. More specific examples of the computer readable medium comprise but are not limited to: an electronic connection (an electronic device) with one or more wires, a portable computer enclosure (a magnetic device), a random access memory (RAM), a read only memory (ROM), an erasable programmable read-only memory (EPROM or a flash memory), an optical fiber device and a portable compact disk read-only memory (CDROM). In addition, the computer readable medium may even be a paper or other appropriate medium capable of printing programs thereon, this is because, for example, the paper or other appropriate medium may be optically scanned and then edited, decrypted or processed with other appropriate methods when necessary to obtain the programs in an electric manner, and then the programs may be stored in the computer memories.

It should be understood that each part of the present disclosure may be realized by the hardware, software, firmware or their combination. In the above embodiments, a plurality of steps or methods may be realized by the software or firmware stored in the memory and executed by the appropriate instruction execution system. For example, if it is realized by the hardware, likewise in another embodiment, the steps or methods may be realized by one or a combination of the following techniques known in the art: a discrete logic circuit having a logic gate circuit for realizing a logic function of a data signal, an application-specific integrated circuit having an appropriate combination logic gate circuit, a programmable gate array (PGA), a field programmable gate array (FPGA), etc.

Those skilled in the art shall understand that all or parts of the steps in the above exemplifying method of the present disclosure may be achieved by commanding the related hardware with programs. The programs may be stored in a computer readable storage medium, and the programs comprise one or a combination of the steps in the method embodiments of the present disclosure when run on a computer.

In addition, each function cell of the embodiments of the present disclosure may be integrated in a processing module, or these cells may be separate physical existence, or two or more cells are integrated in a processing module. The integrated module may be realized in a form of hardware or in a form of software function modules. When the integrated module is realized in a form of software function module and is sold or used as a standalone product, the integrated module may be stored in a computer readable storage medium.

Reference will be made in detail to examples of the present disclosure. It would be appreciated by those skilled in the art that the following examples are explanatory, and cannot be construed to limit the scope of the present disclosure. If the specific technology or conditions are not specified in the examples, a step will be performed in accordance with the techniques or conditions described in the literature in the art (for example, referring to J. Sambrook, et al. (translated by Huang P T), Molecular Cloning: A Laboratory Manual, 3rd Ed., Science Press) or in accordance with the product instructions. If the manufacturers of reagents or instruments are not specified, the reagents or instruments may be commercially available, for example, from Illumina company.

General Method

The method according to embodiments of the present disclosure mainly comprises following steps:

1) noninvasive sampling a pregnant sample containing fetal genetic materials, extracting genomic DNA therefrom;

2) extracting and purifying genomic DNA sample from family members of the fetus, such as parents or grandparents thereof;

3) constructing a sequencing library with every genetic material in accordance with an requirement for different sequencing platform;

4) filtering obtained sequencing data, with filtering criteria based on quality value, adaptor contamination and etc;

5) assembling obtained high-quality sequences as required, aligning an assembled result to a human genome reference sequence, to obtain uniquely-mapped sequences for analyzing using the model.

### Analysis Model

### Marker:

I. the number of sites to be detected is N.
II. haplotypes of parents are respectively recorded as FH={fh₀,fh₁} and MH={mh₀,mh₁},
   in which

mhₖ={m_{1,k}, . . . , m_{i,k}, . . . , m_{N,k}}, fhₖ={f_{1,k}, . . . , f_{i,k}, . . . , f_{N,k}},

∀fh_{i,k},mh_{i,k}∈{A,C,G,T},

k∈{0,1}, i=1,2,3, . . . , N.
III. Unknown fetal haplotype is recorded as H={h₀,h₁}, particularly h₀ and h₁ respectively represent inheriting from mother and father.

h₀={m_{1,x}_{₁}, . . . , m_{i,x}_{ᵢ}, . . . , m_{N,x}_{_{N}}}, h₁={f_{1,y}_{₁}, . . . , f_{i,y}_{ᵢ}, . . . , f_{N,y}_{N}},

in which xᵢ∈{0,1}, yᵢ∈{0,1},
Subscripts xᵢ and yᵢ respectively present sequence pairs, and qᵢ={xᵢ,yᵢ} represents the hidden states which need to be decoded.
While, all hidden states possible presenting constitutes a set Q.
IV. Sequencing data is recorded as S={s₁, . . . , sᵢ, . . . , s_{N}}
   in which sᵢ={n_{i,A},n_{i,C},n_{i,G},n_{i,G}} represents sequencing information of a site, containing the number of four bases, A, C, T and G.
V. A mean fetal concentration and a mean sequencing error rate are respectively recorded as ε and e.
Step 1, constructing a probability distribution vector of an initial state and a transition matrix of haplotypes recombination:
I. The probability distribution of the initial states is recorded as π={πⱼ} (j∈Q).

According to embodiments of the present disclosure, under a circumstance of having no reference data, it may assume that

$${π}_{j} = Pr ( {q}_{1} = j ) {=}^{Δ} \frac{1}{4} , ,$$

i.e., possibilities of each hidden state presenting at the first site are equal.
II. According to embodiments of the present disclosure, a probability of haplotype recombination is recorded as pᵣ=re/N, in which re represents mean times of human gamete recombinations, with a prior data ranging from 25 to 30.
III. According to embodiments of the present disclosure, a transition matrix of haplotypes recombination is recorded as A={aⱼₖ} (j,k∈Q), in which aⱼₖ represents a probability of hidden states transition, i.e.,

$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix} ,\end{matrix}$$

Subscripts xᵢ and yᵢ of fetal haplotypes h₀={m_{1,x}_{₁}, . . . , m_{i,x}_{ᵢ}, . . . , m_{N,x}_{_{N}}} and h₀={f_{1,y}_{₁}, . . . , f_{i,y}_{ᵢ}, . . . , f_{N,y}_{_{N}}} constitute a sequence pair, qᵢ={xᵢ,yᵢ} constitute the hidden states to be encoded. For example, xᵢ=0 represents “in a maternal chromosome, an allele in the corresponding locus is m_{i,0}”.
Step 2, constructing a probability matrix of observations:

According to embodiments of the present disclosure, the probability matrix of observations is recorded as B={b_{i,j}(sᵢ)} (i=1,2,3, . . . , N, j∈Q), in which b_{i,j}(sᵢ) represents “an observed possibility of this sequencing information in a site i, considering maternal haplotype and fetal haplotype (state j, j={xᵢ, yᵢ})”, i.e.,

$$\begin{matrix}{b}_{i , j} ( {s}_{i} ) = Pr ( {s}_{i} \vert {q}_{i} = j , \{ {m}_{0} , {m}_{1} \} ) \\ = \frac{( {n}_{i , A} + {n}_{i , C} + {n}_{i , G} + {n}_{i , T} ) !}{{n}_{i , A} ! {n}_{i , C} ! {n}_{i , G} ! {n}_{i , T} !} \cdot {( {P}_{i , A} )}^{{n}_{i , A}} \cdot {( {P}_{i , C} )}^{{n}_{i , C}} \cdot {( {P}_{i , G} )}^{{n}_{i , G}} \cdot {( {P}_{i , T} )}^{{n}_{i , T}} ,\end{matrix}$$

in which P_{i,base} represents “a possibility of a base in a site i, considering maternal haplotype and fetal haplotype (state j, j={xᵢ, yᵢ})”, i.e.,

$$\begin{matrix}{P}_{i , base} = Pr ( base \vert {q}_{i} = j , \{ {m}_{0} , {m}_{1} \} ) \\ = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \frac{1}{2} ɛ \cdot Δ ( base , {f}_{{y}_{i}} ) ,\end{matrix}$$

in which, an indicator function is

$$Δ ( x , y ) = \{ \begin{matrix}1 - e & x = y \\ e / 3 & x \neq y\end{matrix} .$$

Step 3, constructing a partial probability matrix, and a reversal cursor (taking an example of constructing a one-dimensional probability matrix):

Definition: partial probability

$${δ}_{i} ( {q}_{i} ) = ( {max}_{{q}_{i - 1} ∈ Q} {δ}_{i} ( {q}_{i} ) \cdot {a}_{{q}_{i - 1} {q}_{i}} ) \cdot {b}_{i , {q}_{i}} ( {s}_{i} ) ,$$

Definition: reversal cursor

$${Ψ}_{i} ( {q}_{i} ) = {argmax}_{{q}_{i - 1} ∈ Q} {δ}_{i} ( {q}_{i} ) \cdot {a}_{{q}_{i - 1} {q}_{i}} .$$

Step 4, determining a final state, and tracing back an optional path

Determination of the final state,

$${q}_{N}^{*} = {argmax}_{{q}_{N} ∈ Q} {δ}_{N} ( {q}_{N} ) .$$

The most possible fetal haplotype qᵢ*Ψᵢ(qᵢ) (i=1,2,3, . . . , N−1) is obtained by tracing back the optional path based on the reversal curse.

Step 5, outputting a result

EXAMPLE 1

Sample Collection and Treatment

(1) collected sample included: peripheral blood extracted from a father and a pregnant mother within a family, and fetal umbilical cord blood after birth, all of which were collected in a tube containing EDTA for anticoagulation; saliva were collected from four grandparents using a Oragene® DNA saliva collection/DNA purification kit OG-250.

(2) extracted saliva DNA of the four grandparents were subjected to genotyping using Infinium® HD Human610-Quad BeadChip gene chip.

(3) the peripheral blood collected from the pregnant mother was centrifuged with 1600 g at 4° C. for 10 min, to separate blood cells and plasma. Then obtained plasma was centrifuged with 16000 g at 4° C. for 10 min, to further remove residual leukocytes, to obtain final plasma of the pregnant mother. Then genomic DNA was extracted from the final plasma of the pregnant mother using TIANamp Micro DNA Kit (TIANGEN), to obtain a genomic DNA mixture of mother and fetus thereof. Then maternal genomic DNA was extracted from removed residual leukocytes. Obtained plasma DNA were subjected to library construction based on requirement for HiSeg2000™ sequencer of Illumia® sequencer. Constructed libraries were subjected to a distribution test using Agilent® Bioanalyzer 2100 to meet a requirement for fragment ranges. Then two libraries were subjected to quantification using Q-PCR method. Qualified libraries were subjected to sequencing using Illumina® HiSeg2000® sequencer, with a sequencing cycle of PE101index (i.e., pair-end 101 bp index sequencing), in which parameter settings and operations were based on Illumina® specifications (obtained at http://www.lumina.com/support/documentation.ilmn)

(4) parental peripheral blood, leukocytes extracted from maternal peripheral blood and fetal umbilical cord blood were extracted with their respective genomic DNA using TIANamp Micro DNA Kit (TIANGEN).

Except for plasma DNA sample, all obtained DNA sample needed to be fragmented using Covaris™ to have a length of 500 bp. Obtained DNA fragments and plasma DNA sample were subjected to library construction based on the requirement for HiSeg2000™ sequencer of Illumia® sequencer, with a detailed procedure:

End-repairing reacting system:

| | | |
|---|---|---|
| 10× T4 Polynucleotide kinase buffer | 10 | μL |
| dNTPs (10 mM) | 4 | μL |
| T4 DNA polymerase | 5 | μL |
| Klenow fragments | 1 | μL |
| T4 Polynucleotide kinase | 5 | μL |
| DNA fragments | 30 | μL |

| | |
|---|---|
| ddH₂O | up to 100 μL |

After reacting at 20° C. for 30 min, PCR Purification Kit (QIAGEN) was used in recycling end-repaired products. Then the recycled end-repaired products were finally dissolved in 34 μL of EB buffer.

A reacting system for adding base A at end:

| | | |
|---|---|---|
| 10× Klenow buffer | 5 | μL |
| dATP (1 mM) | 10 | μL |
| Klenow (3′-5′ exo⁻) | 3 | μL |
| DNA | 32 | μL |

After incubating at 37° C. for 30 min, obtained products were purified by MinElute® PCR Purification Kit (QIAGEN) and dissolved in 12 μL of EB buffer, to obtain DNA samples added with base A at end.

Ligating adaptor reacting system:

| | |
|---|---|
| 2× Rapid DNA ligating buffer | 25 μL |
| PEI Adapter oligo-mix (20 μM) | 10 μL |
| T4 DNA ligase | 5 μL |
| DNA sample added with base A at end | 10 μL |

After reacting at 20° C. for 15 min, PCR Purification Kit (QIAGEN) was used in recycling ligated products. The ligated products were finally dissolved in 32 μL of EB buffer.

PCR reacting system:

| | | |
|---|---|---|
| Ligated product | 10 | μL |
| Phusion DNA Polymerase Mix | 25 | μL |
| PCR primer (10 pmol/μL) | 1 | μL |
| Index N (10 pmol/μL) | 1 | μL |
| UltraPureTM Water | 13 | μL |

Reacting procedure was shown as below:

| | | | | |
|---|---|---|---|---|
| 98° C. | 30 | s | | |
| 98° C. | 10 | s | | |
| 65° C. | 30 | s | {close oversize brace} | 10 cycles |
| 72° C. | 30 | s | | |
| 72° C. | 5 | min | | |

| | |
|---|---|
| 4° C. | Hold |

PCR Purification Kit (QIAGEN) was used in recycling PCR products, which were finally dissolved in 50 μL of EB buffer.

Constructed libraries were subjected to a distribution test using Agilent® Bioanalyzer 2100 to meet a requirement for fragment ranges. Then two libraries were subjected to quantification using Q-PCR method. Qualified libraries were subjected to sequencing using Illumina® HiSeg2000™ sequencer, with a sequencing cycle of PE101index (i.e., pair-end 101 bp index sequencing), in which parameter settings and operations were based on Illumina® specifications (obtained at http://www.illumina.com/support/documentation.ilmn)

(5) parental and maternal genomes sequencing genotyping

a. the sequencing data were aligned to a human reference genome (Hg19, NCBI 36.3) using SOAP2.

b. obtained data were subjected to consensus sequence (CNS) construction using SOAPsnp (thousands of planning data were used for Southern Han (CHS) pedigree data).

c. genotypes of a maker site were extracted.

(6) determination of parents' haplotypes

a. constructing a group genotype matrix containing ancestors' and parents' genotypes, i.e., extracting genotypes in the marker site of parents, ancestors and Southern Han pedigree.

b. deducing parents' haplotypes using BEAGLE.

(7) determination of fetal haplotype

a. aligning plasma sequencing data to a human reference genome ((Hg19, NCBI 36.3) using SOAP2;

b. constructing a probability vector of initial states, and a transition matrix of haplotypes recombination,

constructing the probability vector of initial states: taking a model of non-reference data, i.e., probabilities of every initial states were equal, being 0.25.

constructing the transition matrix of haplotypes recombination: conservatively, re=25 (others were same as descriptions in “general method”);

c. calculating sequencing information of each site, and constructing a probability matrix of observations (others were same as descriptions in “general method”);

d. constructing a partial probability matrix, and a reversal curse (others were same as descriptions in “general method”);

e. determining a final state, and tracing back an optional path; and

f. outputting.

According to genotyping results, the accuracy thereof were shown below:

| | | | mother | | | | | | | | |
|---|---|---|---|---|---|---|---|---|---|---|---|
| | | | homozygosis | | | heterozygosis | | | total | | |
| | | | site | accurate | | site | accurate | | site | accurate | |
| | | | number | number | accuracy | number | number | accuracy | number | number | accuracy |
| autosome | father | homozygosis | 199,552 | 199,552 | 100.00% | 66,238 | 63,968 | 96.57% | 265,790 | 263,520 | 99.15% |
| | | heterozygosis | 65,409 | 64,735 | 98.97% | 41,849 | 39,944 | 95.45% | 107,258 | 104,679 | 97.60% |
| | | | 264,961 | 264,287 | 99.75% | 108,087 | 103,912 | 96.14% | 373,048 | 368,199 | 98.70% |

| | | | | | | | | | |
|---|---|---|---|---|---|---|---|---|---|
| chromosome X | 4,881 | 4,881 | 100.00% | 1,718 | 1,478 | 86.03% | 6,599 | 6,359 | 96.36% |

### INDUSTRIAL APPLICABILITY

The method of determining base information of a predetermined region in a fetal genome, the system for determining base information of a predetermined region in a fetal genome and a computer readable medium according to embodiments of the present disclosure may be effectively applied in analyzing the nucleic acid sequence of the predetermined region in the fetal genome.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

Reference throughout this specification to “an embodiment,” “some embodiments”, “one embodiment”, “another example”, “an example”, “a specific example”, or “some examples”, means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example, “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

What is claimed is:

1. A method of determining base information of a predetermined region in a fetal genome, comprising the following steps:
constructing a sequencing library based on a genomic DNA sample of a fetus;
subjecting the sequencing library to sequencing, to obtain a sequencing result of the fetus consisting of a plurality of sequencing data; and
determining the base information of the predetermined region based on the sequencing result of the fetus combining with genetic information of a related individual using a hidden Markov Model.

2. The method of claim 1, wherein the genomic DNA sample of the fetus is extracted from pregnant peripheral blood.

3. The method of claim 1, wherein the sequencing library is subjected to sequencing by at least one selected from Illumina-Solexa, ABI-Solid, Roche-454 and a single molecule sequencing apparatus.

4. The method of claim 1, further comprising a step of aligning the sequencing result of the fetus to a reference sequence, to determine sequencing result deriving from the predetermined region.

5. The method of claim 4, wherein the reference sequence is a human reference genome.

6. The method of claim 1, wherein the related individual is parents of the fetus.

7. The method of claim 1, wherein the step of determining the base information of the predetermined region using the hidden Markov Model is performed based on Viterbi algorithm.

8. The method of claim 7, wherein in the Viterbi algorithm, 0.25 is used as a probability distribution of an initial status, re/N is used as a recombination probability, with re being 25˜30, preferably re being 25, and N being a length of the predetermined region,
$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix}\end{matrix}$$
is used as a recombination transition matrix with pᵣ being re/N.

9. The method of claim 4, wherein the step of aligning the sequencing result of the fetal genome to the reference sequence to determine sequencing result deriving from the predetermined region further comprises:
determining a base having the highest probability based on a formula of
$${P}_{i , base} = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \frac{1}{2} ɛ \cdot Δ ( base , {f}_{{y}_{i}} )$$
$$wherein$$
$$Δ ( x , y ) = \{ \begin{matrix}1 - e & x = y \\ e / 3 & x \neq y\end{matrix} .$$

10. The method of claim 1, wherein the predetermined region is a site previously determined as having a genetic polymorphism.

11. The method of claim 10, wherein the genetic polymorphism is at least one selected from single nucleotide polymorphism and STR.

12. A system for determining base information of a predetermined region in a fetal genome, comprising:
a library constructing apparatus, adapted for constructing sequencing library based on a genomic DNA sample of a fetus;
a sequencing apparatus, connected to the library constructing apparatus, and adapted for subjecting the sequencing library to sequencing, to obtain a sequencing result of the fetus consisting of a plurality of sequencing data; and
an analyzing apparatus, connected to the sequencing apparatus, and adapted for determining the base information of the predetermined region based on the sequencing result of the fetus combining with genetic information of a related individual using a hidden Markov Model.

13. The system of claim 12, further comprising a DNA sample extracting apparatus, adapted for extracting the genomic DNA sample of the fetus from pregnant peripheral blood.

14. The system of claim 12, the sequencing apparatus is at least one selected from Illumina-Solexa, ABI-Solid, Roche-454 and a single molecule sequencing apparatus.

15. The system of claim 12, further comprising an aligning apparatus, connected to the sequencing apparatus, and adapted for aligning the sequencing result of the fetus to a reference sequence, to determine sequencing result deriving from the predetermined region.

16. The system of claim 12, wherein the analyzing apparatus is adapted for determining the base information of the predetermined region using a hidden Markov Model based on Viterbi algorithm.

17. The system of claim 16, wherein in the Viterbi algorithm, 0.25 is used as a probability distribution of an initial status, re/N is used as a recombination probability, with re being 25˜30, preferably re being 25, and N being a length of the predetermined region,
$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix}\end{matrix}$$
is used as a recombination transition matrix with pᵣ being re/N.

18. The system of claim 15, wherein the aligning apparatus is adapted for determining a base having the highest probability based on a formula of
$${P}_{i , base} = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \frac{1}{2} ɛ \cdot Δ ( base , {f}_{{y}_{i}} )$$
$$wherein$$
$$Δ ( x , y ) = \{ \begin{matrix}1 - e & x = y \\ e / 3 & x \neq y\end{matrix} .$$

19. A computer readable medium comprising a plurality of instructions, adapted for determining base information of a predetermined region based on a sequencing result of a fetus combining with genetic information of a related individual using a hidden Markov Model.

20. The computer readable medium of claim 19, wherein the plurality of instructions are adapted for determining the base information of the predetermined region using the hidden Markov model based on Viterbi algorithm.

21. The computer readable medium of claim 20, wherein in the Viterbi algorithm, 0.25 is used as a probability distribution of an initial status, re/N is used as a recombination probability, with re being 25˜30, preferably re being 25, and N being a length of the predetermined region,
$$\begin{matrix}{a}_{jk} = Pr ( {q}_{i} = k \vert {q}_{i - 1} = j ) \\ = \{ \begin{matrix}{( 1 - {p}_{r} )}^{2} & {x}_{i} = {x}_{i - 1} , {y}_{i} = {y}_{i - 1} \\ ( 1 - {p}_{r} ) \cdot {p}_{r} & {x}_{i} = {x}_{i - 1} , {y}_{i} \neq {y}_{i - 1} or {x}_{i} \neq {x}_{i - 1} , {y}_{i} = {y}_{i - 1} \\ {p}_{r}^{2} & {x}_{i} \neq {x}_{i - 1} , {y}_{i} \neq {y}_{i - 1}\end{matrix}\end{matrix}$$
is used as a recombination transition matrix with pᵣ being re/N.

22. The computer readable medium of claim 19, wherein the plurality of instructions are adapted for aligning the sequencing result of the fetus to a reference sequence, to determine sequencing result deriving from the predetermined region.

23. The computer readable medium of claim 22, wherein the plurality of instructions are further adapted for determining a base having the highest probability based on based on a formula of
$${P}_{i , base} = {\sum }_{k ∈ \{ 0 , 1 \}} \frac{1}{2} ( 1 - ɛ ) Δ ( base , {m}_{k} ) + \frac{1}{2} ɛ \cdot Δ ( base , {m}_{{x}_{i}} ) + \frac{1}{2} ɛ \cdot Δ ( base , {f}_{{y}_{i}} )$$
$$wherein$$
$$Δ ( x , y ) = \{ \begin{matrix}1 - e & x = y \\ e / 3 & x \neq y\end{matrix} .$$

24. The computer readable medium of claim 19, wherein the predetermined region is a site previously determined as having a genetic polymorphism.

25. The computer readable medium of claim 24, wherein the genetic polymorphism is at least one selected from single nucleotide polymorphism and STR.
